SEQUENCE LISTING (5 sequences)

SEQ ID NO: 1 - protein, 19 aa, Homo sapiens
LQSGTHCLWTDQLLQGSEK

SEQ ID NO: 2 - protein, 16 aa, Homo sapiens
FVYTPAMESVCGYFHR

SEQ ID NO: 3 - protein, 11 aa, Homo sapiens
EPGLCTWQSLR

SEQ ID NO: 4 - protein, 12 aa, Homo sapiens
GFQALGDAADIR

SEQ ID NO: 5 - protein, 9 aa, Homo sapiens
SEEFLIAGK

(19) United States
(12) Patent Application Publication
(10) Pub. No.: US 2014/0335535 A1
(43) Pub. Date: Nov. 13, 2014

(54) PEPTIDE MARKER FOR CANCER DIAGNOSIS AND CANCER DIAGNOSIS METHOD USING THE SAME

(71) Applicant: Korea Basic Science Institute, Daejeon (KR)

(72) Inventors: Yeong Hee AHN, Chungcheongbuk-do (KR); Jong Shin YOO, Seoul (KR)

(73) Assignee: Korea Basic Science Institute, Daejeon (KR)

(21) Appl. No.: 14/205,974

(22) Filed: Mar. 12, 2014

Related U.S. Application Data
(63) Continuation-in-part of application No. 13/375,527, filed on Dec. 1, 2011, now abandoned.

(30) Foreign Application Priority Data
Dec. 29, 2009 (KR) 1020090133078

(51) Int. Cl. G01N 33/574

(52) U.S. Cl. CPC G01N 33/57423
USPC 435/7.1

(57) ABSTRACT

The present invention relates to a peptide marker for cancer diagnosis and a cancer diagnosis method using the same, more precisely to a peptide marker for cancer diagnosis screened by the following steps: separating and concentrating glycoproteins including a certain glycan chain related to the occurrence of cancer; hydrolyzing the glycoproteins to obtain polypeptides; and quantitatively analyzing the polypeptides to identify certain polypeptides that can track quantitative changes in glycoproteins glycosylated in a specific manner by the occurrence of cancer, and to a cancer diagnosis method using the said peptide marker.

### CROSS-REFERENCE TO RELATED APPLICATIONS

This application is a continuation-in-part of U.S. patent application Ser. No. 13/375,527, filed Dec. 1, 2011; which is a 371 national phase filing of PCT/KR10/08666, filed Dec. 6, 2010; which claims the benefit of Korean Patent Application No. 1020090133078, filed Dec. 29, 2009; the entire disclosure of each of which is hereby incorporated herein by reference.

### BACKGROUND OF THE INVENTION

1. Field of the Invention

The present invention relates to a cancer diagnosis method using certain polypeptides that can track quantitative changes in glycoproteins glycosylated in a specific manner by the occurrence of cancer.

2. Description of the Related Art

Proteins are involved in various life-supporting activities and post-translationally modified by signal transmission whenever necessary. The most representative post-translational modification processes are glycosylation and phosphorylation. In particular, regarding glycosylation of a glycoprotein, many monosaccharides existing on the surface of cell membrane are passed through the cell membrane by signal transduction, leading to glycosylation of a required protein by N-acetylglucosaminyltransferase. Such glycoprotein plays an important role as being located on the outer membrane. Once glycoproteins finish their required role, they proceed to glucolysis by glycosidase. However, many glycoproteins or glycolipids located on the surface of cell membrane often experience aberrant glycosylation by the specific signal such as oncogene, etc. Many diseases have been known to be closely related to such abnormal functions of glycosidase and glycosyltransferase triggered by abnormal signal transduction by oncogene (Kim, Y. J., et al., Glycoconj. J., 1997, 14, 569-576., Hakomori, S., Adv. Cancer Res., 1989, 52, 257-331., Hakomori, S., Cancer Res., 1996, 56, 5309-5318).

In the aberrant glycosylation pattern observed in cancer cells, changes in the size of N-linked glycosylation, the number of side chains, and the increase of sialylation and fucosylation are observed along with the changes in the size of glycan chain by polylactosamine formation. Such glycoprotein, thus, can be used as a cancer marker for the identification of cancer and confirmation of cancer progression by taking advantage of the said pattern observed in the aberrant glycosylation. Such aberrant glycosylation affects functions of the glycosylated protein such as folding, recognition, and solubility, etc by the specificity of the structure and the size of glycan chain (Varki, A. et al., Glycobiology 1993, 3, 97-130., Parodi, A. J. et al., Annu. Rev. Biochem. 2000, 69, 69-93). In particular, such glycotransferase as N-acetylglucosaminyltransferase that is abnormally activated in cancer cells induces the aberrant glycosylation which facilitates the distinguishment of cancer cells from normal cells, suggesting that the aberrantly glycosylated glycoprotein can be used as a marker for cancer diagnosis (Dennis, J. W.; Laferte, S.; Waghorne, C.; Breitman, M. L.; Kerbel, R. S. Beta 1-6 branching of Asn-linked oligosaccharides is directly associated with metastasis. Science 1987, 236: 582-585). The glycoprotein harboring information on cancer is secreted to extracellular media once it completes its role therein, or is fallen off the cell membrane and shed to media. Therefore, diverse culturing media for cancer cells, cancer tissue lysis, and patient's blood samples can be proper materials for detection of the glycoprotein containing information on cancer, which can be used as a marker for cancer diagnosis.

Difference of glycosylation in protein samples obtained from the normal group and the cancer patient group can be an important clue to distinguish patient group from normal group. Up to date, many analysis methods have been reported to tell the difference of a glycoprotein. Glycan profiling is an example of the methods to screen difference in glycosylation of a protein, which analyzes glycans obtained from hydrolysis of glycoproteins by using mass spectrometer (Barkauskas. et al., Bioinformatics, 2009, 25, 251-257). However, this method has a disadvantage of losing information on the glycosylated isoform in relation to characteristics of glycosylation and glycosylated structure in the specific glycosylation site of each protein because this method only enables mass analysis of numbers of glycosylated isoform mixtures composed of all different isoforms originated from different proteins and glycosylation sites but having equal weights.

Glyco-capturing method using hydrazide has been used for the enrichment of a glycoprotein (Zhang H. et al., Nat. Biotechnol., 2003, 21, 660˜666). However, this method has a disadvantage of losing information on specific structured aberrant glycosylation derived from cancer cells because intact glycosylation structure cannot be maintained during glycan-capturing.

To separate and enrich a glycoprotein or a glycopeptide, considering different structures of intact glycan chain, ConA(Concanavalin A), WGA(Wheat germ agglutinin), Jacalin, SNA(Sambucus nigra agglutinin), AAL(Aleuria aurantia lectin), L-PHA(Phytohemagglutinin-L), PNA(Peanut agglutinin), LCA(Lens culimaris agglutinin-A), ABA(Agaricus biflorus agglutinin), DBA(Dolichos biflorus agglutinin), DSA(Datura stramonium agglutinin), ECA(Erythrina cristagalli agglutinin), SBA(Soybean agglutinin), SSA(Sambucus sieboldiana agglutinin), UEA(Ulex europaeus agglutinin), VVL(Vicia villosa lectin), BPL(Bauhinia purpurea lectin), or multilectin prepared by combinations of the above lectins can be used (Yang, Z. et al., J. Chromatogr, A, 2004, 1053, 79-88., Wang, Y. et al., Glycobiology, 2006, 16, 514-523). This is the method to utilize the selectivity of lectin to glycan chain structure of a glycoprotein, so that selective separation and enrichment of a glycoprotein having specific glycan chain structure can be possible. In particular, complexity of the target sample can be significantly reduced by eliminating such proteins that do not show affinity to lectin through the separation process of glycoproteins selective to lectin. The separated and enriched glycoproteins can be analyzed qualitatively and quantitatively by using various analysis methods.

One of those methods for analyzing a glycoprotein by using the selectivity of lectin to glycan chain structure of a glycoprotein is lectin-blotting. This method is generally performed along with immunoblotting characterized by high selectivity against certain proteins. At this method, an antibody against the antigen protein is required. If the corresponding antibody is not prepared, the protein cannot be analyzed by this method. Lectin-blotting utilizes gel-separation technique, suggesting that there is still a disadvantage of limited analyzing speed and questions remain on liability in quantitative analysis. When array technique using an antibody and lectin is used, analyzing speed and sensitivity can be significantly improved, compared with the conventional lectin-blotting (Forrester, S. et. al., Low-volume, high-throughput sandwich immunoassays for profiling plasma proteins in mice: identification of early-stage systemic inflammation in a mouse model of intestinal cancer. Mol Oncol 2007, 1(2): 216-225). However, this technique requires a liable antibody and it is very difficult to obtain each antibody against every glycoprotein newly identified at a massive scale.

In the meantime, mass spectrometry is a very useful method for high-speed, high-sensitivity qualitative and quantitative analysis of very complicated proteome samples. In particular, multiple reaction monitoring is the method to analyze quantitatively, with high liability, polypeptides having comparatively small molecular weights which are usually generated by hydrolysis of a protein. This method is especially useful when an antibody against the target protein cannot be obtained (Kuhn, E. et. al., Quantification of C-reactive protein in the serum of patients with rheumatoid arthritis using multiple reaction monitoring mass spectrometry and ¹³C-labeled peptide standards. Proteomics 2004, 4(4): 1175-1186). MRM is an analysis method with high sensitivity that facilitates selective analysis of the target peptide from very complicated samples by at least one of liquid chromatography and two times of precursor mass selection and fragment ion selection from those peptides generated by hydrolysis of the target protein (Anderson L, et al., Mol. Cell Proteomics. 2006, 5, 573-588).

The sample like plasma proteome is composed of at least 50,000 constituents and the density of protein components is very dynamic (1˜10¹², pg/ml). So, a biomarker candidate protein existing at a very low concentration is hard to detect and analyze quantitatively by liquid chromatography-mass spectrometry (LC/MS/MS)(Anderson N. L. et al., Mol. Cell Proteomics. 2002, 1, 845-867). To minimize complexity of the sample for efficient detection of a disease biomarker in plasma, proteins taking about 90% or more of plasma proteome such as albumin, IgG, IgA, transferrin and haptoglobin can be first eliminated. And then the resultant proteome is used for analysis. Even after the minimization of the complexity of the sample by eliminating those proteins exiting at high concentrations in plasma and the increased high selectivity against the target peptide by LC-MRM, if a target marker protein exists at a very low concentration in the sample, the concentration of the marker protein or the hydrolyzed marker peptide is increased through enrichment process of the marker protein or the marker peptide by using immunoaffinity to improve LOD (limit of detection) and LOQ (limit of qualification).

The present inventors have tried to develop a method for cancer diagnosis using quantitative information on specific glycosylation occurring in a glycoprotein in relation to cancer development. As a result, the inventors screened marker peptides by the following steps: separating and concentrating glycoproteins including a certain glycan chain related to the occurrence of cancer; hydrolyzing the glycoproteins to obtain polypeptides; and quantitatively analyzing the polypeptides to identify certain polypeptides that can track quantitative changes in glycoproteins glycosylated in a specific manner by the occurrence of cancer. The present inventors further completed this invention by confirming that the said marker peptides can be effectively used for cancer diagnosis.

### SUMMARY OF THE INVENTION

It is an object of the present invention to provide a method for cancer diagnosis using specific polypeptides that can tract quantitative changes in glycoproteins glycosylated in a specific manner by the occurrence of cancer.

It is another object of the present invention to provide a kit for cancer diagnosis comprising a specific antibody against the specific polypeptide that can tract quantitative changes in glycoproteins glycosylated in a specific manner by the occurrence of cancer.

It is also an object of the present invention to provide a kit for cancer diagnosis comprising a specific antibody specifically binding to the specific polypeptide that can tract quantitative changes in glycoproteins glycosylated in a specific manner by the occurrence of cancer.

It is further an object of the present invention to provide a biochip for cancer diagnosis comprising a biomolecule specifically binding to the specific polypeptide that can tract quantitative changes in glycoproteins glycosylated in a specific manner by the occurrence of cancer.

To achieve the above objects, the present invention provides a method to provide information for cancer diagnosis comprising the following steps:

1) separating and concentrating glycoproteins by treating a sample obtained from a subject with lectin;

2) hydrolyzing the glycoproteins of step 1) to obtain polypeptides;

3) quantitatively analyzing the polypeptides of step 2); and

4) diagnosing cancer or high risk of cancer when the subject has at least one of those polypeptides selected from the group consisting of those polypeptides having the molecular weights of 2143.0, 1905.9, 1288.6, 1232.6 and 992.5 confirmed by the quantitative analysis of step 3).

The present invention also provides a method to provide information for cancer diagnosis comprising the following steps:

1) separating and concentrating glycoproteins by treating a sample obtained from a subject with lectin;

2) hydrolyzing the glycoproteins of step 1) to obtain polypeptides;

3) sequencing and quantitatively analyzing the polypeptides of step 2); and

4) diagnosing cancer or high risk of cancer when the subject has at least one of those polypeptides selected from the group consisting of those polypeptides having the amino acid sequences represented by SEQ. ID. NO: 1-NO: 5 confirmed by the sequencing and quantitative analysis of step 3).

The present invention also provides a kit for cancer diagnosis containing an antibody or a combination of antibodies specifically binding to the polypeptide having one of the amino acid sequences each represented by SEQ. ID. NO: 1-NO: 5.

The present invention also provides a biochip for cancer diagnosis on which a biomolecule specifically binding to the polypeptide having one of the amino acid sequences each represented by SEQ. ID. NO: 1-NO: 5 is integrated on the solid substrate.

The present invention also provides a use of the antibody or the combination of antibodies specifically binding to the polypeptide having one of the amino acid sequences each represented by SEQ. ID. NO: 1-NO: 5 for the preparation of a kit for cancer diagnosis.

In addition, the present invention provides a use of the biomolecule specifically binding to the polypeptide having one of the amino acid sequences each represented by SEQ. ID. NO: 1-NO: 5 for the preparation of a biochip for cancer diagnosis.

### Advantageous Effect

The present invention provides a method to distinguish effectively the cancer patient group from the normal healthy group by performing quantitative analysis of the marker glycoprotein isoform having a cancer specific glycan chain structure that is quantitatively changed sensitively by N-acetylglucosaminyltransferase, the glycosyltransferase overexpressed in many types of cancer cells. The method of the present invention facilitates fast and simple diagnosis of cancer from the sample of a subject by performing quantitative analysis of the hydrolyzed marker peptide containing the information on the amount of the marker glycoprotein isoform having a cancer specific glycan chain structure. At this time, the selected peptide can be effectively used as a marker for cancer diagnosis.

### BRIEF DESCRIPTION OF THE DRAWINGS

The application of the preferred embodiments of the present invention is best understood with reference to the accompanying drawings, wherein:

FIG. 1 is a diagram illustrating the quantitative analysis result of the marker glycoprotein having a cancer specific glycan chain structure by using the marker peptide representative after performing lectin-separation with both GnT-V overexpressing group (GnT-V treated) and normal control group. In the GnT-V treated group, the expression of the marker glycoprotein having a cancer specific structure was significantly increased (approximately 12.5 times higher), proving high specificity.

FIG. 2 is a diagram illustrating the quantitative analysis result of the total marker glycoprotein expressed in both GnT-V overexpressing group (GnT-V treated) and normal control group by using the marker peptide representative without lectin-separation, proving low specificity.

FIG. 3 is a diagram showing the chromatogram illustrating the transition of the marker peptide represented by SEQ. ID. NO: 4 obtained by LC/MRM using blood samples of the normal control group.

FIG. 4 is a diagram showing the chromatogram illustrating the transition of the marker peptide represented by SEQ. ID. NO: 4 obtained by LC/MRM using blood samples of the colon cancer patient group.

FIG. 5 is a diagram illustrating the data of Table 3 explaining the results of repeated LC/MRM, in which the concentration of the marker peptide is approximately 4.3 times higher in the colon cancer patient group than that in the normal control group.

FIGS. 6A-D show chromatograms illustrating the transition of the marker peptide represented by SEQ. ID. NO: 4 obtained by LC/MRM MS using blood samples of the adenocarcinoma lung cancer (ADLC)[FIG. 6B], squamous-cell carcinoma lung cancer (SQLC) [FIG. 6C] and small-cell lung cancer (SCLC) [FIG. 6D] patient group.

FIG. 7 is a diagram illustrating the data of Table 4 explaining the results of repeated LC/MRM, in which the concentration of the marker peptide is approximately 4.4˜5.08 times higher in the adenocarcinoma lung cancer (ADLC), squamous-cell carcinoma lung cancer (SQLC) patient group than that in the normal control group.

### DESCRIPTION OF THE PREFERRED EMBODIMENTS

Hereinafter, the present invention is described in detail.

The present invention provides a method for cancer diagnosis using information on specific glycosylation of a glycoprotein.

Particularly, the present invention provides a method for cancer diagnosis by using one or more peptide markers selected by the following steps: separating glycoproteins containing specific glycan chain related to cancer development from a sample by using lectin; hydrolyzing the separated glycoproteins to give peptides; and selecting marker peptides among the peptide samples obtained by hydrolysis above that can track quantitative changes in glycoproteins glycosylated in a specific manner by the occurrence of cancer.

In a preferred embodiment of the present invention, it is preferred to diagnose cancer by the method comprising the following steps, but not always limited thereto:

1) separating and concentrating glycoproteins by treating a sample obtained from a subject with lectin;

2) hydrolyzing the glycoproteins of step 1) to obtain polypeptides;

3) quantitatively analyzing the polypeptides of step 2); and

4) diagnosing cancer or high risk of cancer when the subject has at least one of those polypeptides selected from the group consisting of those polypeptides having the molecular weights of 2143.0, 1905.9, 1288.6, 1232.6 and 992.5 confirmed by the quantitative analysis of step 3).

In another preferred embodiment of the present invention, it is preferred to diagnose cancer by the method comprising the following steps, but not always limited thereto:

1) separating and concentrating glycoproteins by treating a sample obtained from a subject with lectin;

2) hydrolyzing the glycoproteins of step 1) to obtain polypeptides;

3) sequencing and quantitatively analyzing the polypeptides of step 2); and

4) diagnosing cancer or high risk of cancer when the subject has at least one of those polypeptides selected from the group consisting of those polypeptides having the amino acid sequences represented by SEQ. ID. NO: 1-NO: 5 confirmed by the sequencing and quantitative analysis of step 3).

In another preferred embodiment of the present invention, it is preferred to diagnose lung cancer by the method comprising the following steps, but not always limited thereto:

a) contacting blood from a test subject with one or more lectins;

b) hydrolyzing glycoproteins that bind one or more lectins with one or more enzymes to produce peptides derived from a glycoprotein, wherein the hydrolysis is conducted in solution;

c) concentrating the glycoprotein-derived hydrolysate of step b) by contacting with a peptide-selective monoclonal antibody or a combination of peptide-selective antibodies that specifically binds a peptide derived from the glycoprotein of step b);

d) analyzing the antibody-bound peptides from step c) to determine the level of target peptides having the amino acid sequences represented by SEQ ID NO:1, SEQ ID NO:2, SEQ ID NO:3, SEQ ID NO:4, and SEQ ID NO:5 wherein the target peptides were derived from a glycoprotein TIMP1 having a cancer cell-specific glycan chain; and

e) identifying the test subject as having lung cancer or having a high risk for developing lung cancer when the level of the target peptides from step d) is greater than the level of the corresponding target peptides in a normal subject.

In another preferred embodiment of the present invention, it is preferred to diagnose lung cancer by the method comprising the following steps, but not always limited thereto:

a) contacting blood from a test subject with one or more lectins;

b) hydrolyzing glycoproteins that bind one or more lectins with one or more enzymes to produce peptides derived from a glycoprotein, wherein the hydrolysis is conducted in solution;

c) concentrating the glycoprotein-derived hydrolysate of step b) by contacting with a peptide-selective monoclonal antibody or a combination of peptide-selective antibodies that specifically binds a peptide derived from the glycoprotein of step b);

d) analyzing the antibody-bound peptides from step c) to determine the level of target peptides having the amino acid sequences represented by SEQ ID NO:1, SEQ ID NO:2, SEQ ID NO:3, SEQ ID NO:4, and SEQ ID NO:5 wherein the target peptides were derived from a glycoprotein TIMP1 having a cancer cell-specific glycan chain; and

e) identifying the test subject as having lung cancer or having a high risk for developing lung cancer when the level of the target peptides from step d) is greater than the level of the corresponding target peptides in a normal subject.

In another preferred embodiment of the present invention, it is preferred to identify adenocarcinoma lung cancer subtype or squamous-cell carcinoma lung cancer subtype from the small-cell lung cancer subtype by the method comprising the following steps, but not always limited thereto:

1) contacting blood from a test subject with one or more lectins;

2) hydrolyzing glycoproteins that bind one or more lectins with one or more enzymes to produce peptides derived from a glycoprotein, wherein the hydrolysis is conducted in solution;

3) concentrating the glycoprotein-derived hydrolysate of step 2) by contacting with a peptide-selective monoclonal antibody or a combination of peptide-selective antibodies that specifically binds a peptide derived from the glycoprotein of step 2);

4) analyzing the antibody-bound peptides from step 3) to determine the level of target peptides having the amino acid sequences represented by SEQ ID NO:1, SEQ ID NO:2, SEQ ID NO:3, SEQ ID NO:4, and SEQ ID NO:5 wherein the target peptides were derived from a glycoprotein TIMP1 having a cancer cell-specific glycan chain; and

5) identifying the test subject as having adenocarcinoma lung cancer subtype or squamous-cell carcinoma lung cancer subtype from the small-cell lung cancer subtype or having a high risk for developing adenocarcinoma lung cancer subtype or squamous-cell carcinoma lung cancer subtype from the small-cell lung cancer subtype when the level of the target peptides from step 4) is greater than the level of the corresponding target peptides in a normal subject.

In this invention, the sample is the one that can be obtained from living things harboring proteins containing information related to cancer development and progress, which is exemplified by biological tissue, cell line or culture fluid originated from culture of the biological tissue, saliva, and blood. Particularly, the glycoprotein harboring information on cancer is secreted to extracellular media once it finishes playing its role, or is fallen off the cell membrane and shed to media. Therefore, culturing media of various cancer cell lines and patients' blood samples are good samples for the screening of glycoproteins containing information on cancer, which are cancer markers. In the case of blood sample, difference of concentrations of each protein component existing in blood is very huge. So, pre-treatment is preferred to minimize the complexity of a sample by using protein removal column (ex, MARS, Multiple Affinity Removal System), but not always limited thereto.

In this invention, ‘cancer specific glycosylation of a glycoprotein’ means that glycosylation of a protein is induced abnormally in cancer patients and those having history of cancer. Such specific glycosylation can occur at specific glycan chain sites connected to glycosylation sites such as asparagine, threonin, or serine site. The glycan chain having a cancer specific structure shows glycan microheterogeneity by sharing one glycosylation site with the glycan chain with normal structure. Thus, the said specific glycan chain exists among many glycan-isoforms in one glycosylation site at a very low concentration, nonstoichiometrically to the protein. Therefore, to measure the quantitative changes of such specific glycan chain accurately, it is preferred to separate and concentrate the specific glycan chain from various glycan-isoforms, but not always limited thereto.

In this invention, lectin can be used to separate and concentrate the isoform having specific glycan chain from many glycan-isoforms having different glycan chain structures. This is the method to take advantage of selectivity of lectin against glycan chain structure of a glycoprotein, so that it facilitates selective separation and concentration of marker glycoproteins having a specific glycan chain structure. According to the glycan chain structure, a variety of lectins such as ConA, WGA, Jacalin, SNA, AAL, L-PHA, PNA, LCA, ABA, DBA, DSA, ECA, SBA, SSA, UEA, VVL, or BPL can be used either alone or together as a combination for the separation and concentration. Various lectins can be used for the separation of proteins having different glycan-isoforms selectively from the total samples.

In a preferred embodiment of the present invention, the glyco-isoform having glycan chain with specific structure was separated and concentrated by using L-PHA to trace the changes of specific glycosylation (increase of side chain of glycan chain by the connection of β-1,6-GlcNAc) mediated by N-acetylglucosaminyltransferase, the glycosyltransferase overexpressed in many types of cancer cells. At this time, many proteins not showing affinity to L-PHA were eliminated during the separation procedure of L-PHA selective glycoproteins, suggesting that the complexity of a target sample was significantly decreased.

In this invention, it is preferred to hydrolyze the high molecular weight proteins separated by using lectin into peptide fragments having smaller molecular weight to increase efficiency of analysis. To obtain peptides by hydrolyzing glycoproteins, a biological method using various hydrolases or a chemical method using a chemical reagent inducing hydrolysis in a specific amino acid site can be used. At this time, one or more hydrolases selected from the group consisting of Arg-C, Asp-N, Glu-C, Lys-C, chymotrypsin and trypsin can be used, and trypsin is more preferred, but not always limited thereto. To enhance hydrolysis efficiency and efficiency in analysis of generated peptides, pre-treatment of samples such as denaturation, reduction, and cystein alkylation can be performed before hydrolysis.

In this invention, target peptides (marker peptides) that can track quantitative changes in marker glycoproteins glycosylated in a specific manner by the occurrence of cancer can be selected by comparative quantitative analysis of the hydrolyzed peptide samples obtained from normal and patient groups. In particular, culturing media of diverse cancer cell lines and patient's blood samples are good samples for the screening of the cancer marker that is the glycoprotein containing information on cancer. Herein, the said cancer includes every kind of cancer inducing cancer specific glycosylation, which is exemplified by colon cancer, liver cancer, stomach cancer, lung cancer, uterine cancer, breast cancer, prostatic cancer, thyroid cancer, and pancreatic cancer, adenocarcinoma lung cancer (ADLC), squamous-cell carcinoma lung cancer (SQLC) and small-cell lung cancer (SCLC, etc.

In a preferred embodiment of the present invention, marker glycoprotein candidates showing quantitative changes by certain glycosylation specifically induced by N-acetylglucosaminyltransferase, the glycosyltransferase overexpressed in many types of cancer cells, were selected. Then, quantitative mass spectrometry was performed with the candidate marker peptides. Finally, the method for cancer diagnosis with the selected peptide marker was completed by confirming the cancer marker peptides affected by N-acetylglucosaminyltransferase, the glycosyltransferase overexpressed in human colon cancer cells (see Table 1).

In this invention, the marker peptides selected from the peptide samples obtained by hydrolysis after concentration using lectin can be composed of one or more peptides originated from a single glycoprotein, or can include peptides originated from different glycoproteins. Therefore, the selected marker peptides can be used for sample analysis together with at least two peptides if necessary.

To analyze the hydrolyzed peptides including marker peptides quantitatively, immune-precipitation or immunoblotting using a peptide-specific antibody can be used along with mass spectrometry in this invention. In particular, mass spectrometry has no limitation in peptides. This method has another advantage of high speed/high sensitivity. Quantitative analysis method performed by labeling peptides with an isotope-labeled marker (iTRAQ, ICAT etc.) or multiple reaction monitoring (MRM) performed by adding stable isotope standard to the sample as an internal standard can be used. In a preferred embodiment of the present invention, MRM was performed by adding an isotope-labeled marker peptide standard to the sample as an internal standard to quantitatively analyze cancer marker peptides affected by N-acetylglucosaminyltransferase, the glycosyltransferase overexpressed in cancer cells.

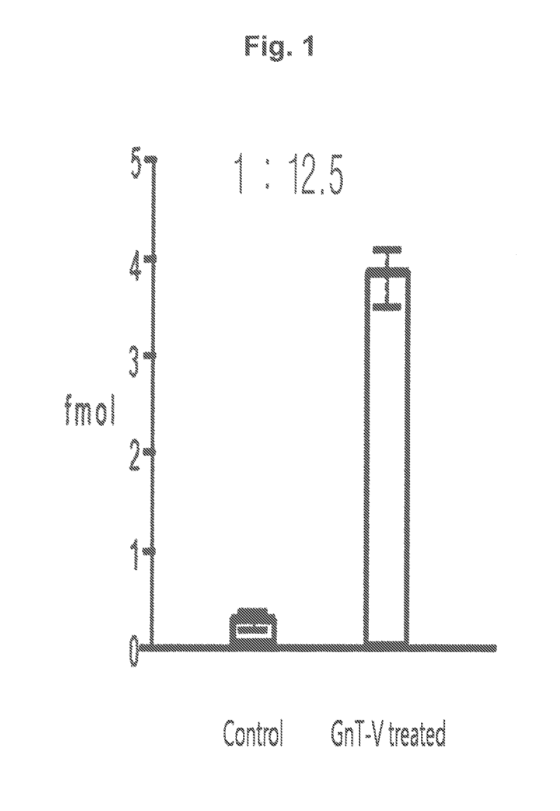

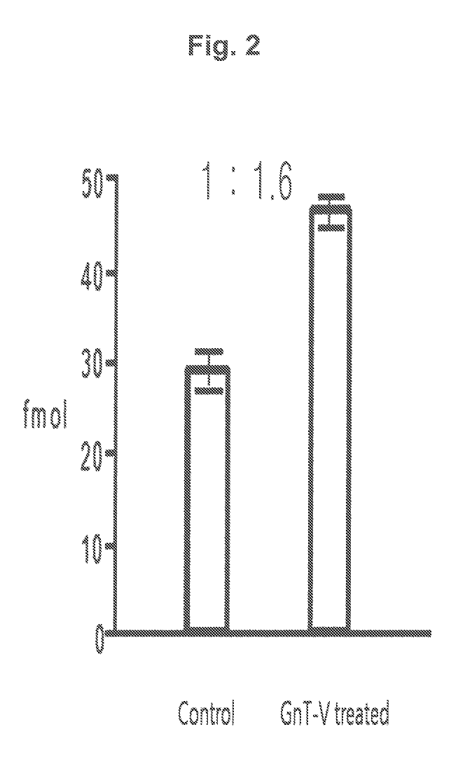

In a preferred embodiment of the present invention, the marker peptide having the peptide mass of 1232.6 was selected among the marker peptides (see Table 1) to investigate the effect of N-acetylglucosaminyltransferase V (GnT-V), the glycosyltransferase overexpressed in many types of cancer cells, on specific glycosylation of a marker glycoprotein (see FIGS. 1 and 2). Glycan-isoforms having a specific glycan chain structure were separated by using lectin from the GnT-V overexpressing cell line (GnT-V-treated), and from the GnT-V not-overexpressing cell line (control), followed by MRM via hydrolysis. As a result, as shown in FIG. 1, the generation of glycan-isoform having a specific glycan-chain structure with high affinity selectively to L-PHA was remarkably increased in the sample where GnT-V was over-expressed. To confirm that the above result was attributed to the generation of glycan-isoform having a specific glycan-chain structure, the same sample was hydrolyzed without using lectin, followed by MRM using the same marker peptide. In this case, that is in the case that separation process of glycan-isoform having a specific glycan-chain structure by using lectin is skipped, the amount of the measured marker peptide represents total amount of the marker glycoprotein including all of glycan-isoforms in the GnT-V overexpressing sample (GnT-V-treated) and in the control. As a result, as shown in FIG. 2, the expression level of the total marker glycoprotein was not much different between the two groups; the GnT-V overexpressing group (GnT-V-treated) and the control. That is, the total expression level of the marker glycoprotein represented by the marker peptide having the peptide mass of 1232.6 among the marker peptides (see Table 1) was not affected significantly by GnT-V overexpression (without separation using lectin, see FIG. 2). In the meantime, the expression level of glycan-isoform having a specific glycan chain structure was significantly affected by GnT-V overexpression (with separation using lectin, see FIG. 1).

Therefore in this invention, mass spectrometry was performed only with the marker glycoprotein isoform having a specific glycan chain structure showing quantitative changes sensitively by N-acetylglucosaminyltransferase, the glycosyltransferase overexpressed in many types of cancer cells, indicating that the marker of the invention can be effectively used for the distinguishment of cancer patient group from normal group. Selective separation of the glycan-isoform having a specific glycan chain structure from all different kinds of glycan-isoforms of marker glycoproteins can be achieved by using proper lectin. Quantitative analysis of the glycan-isoform having a specific glycan-chain structure can be performed by analyzing the hydrolyzed marker peptide derived from the marker glycoprotein. Theoretically, all the peptides hydrolyzed during the hydrolysis of the marker glycoprotein can be used as marker peptides.

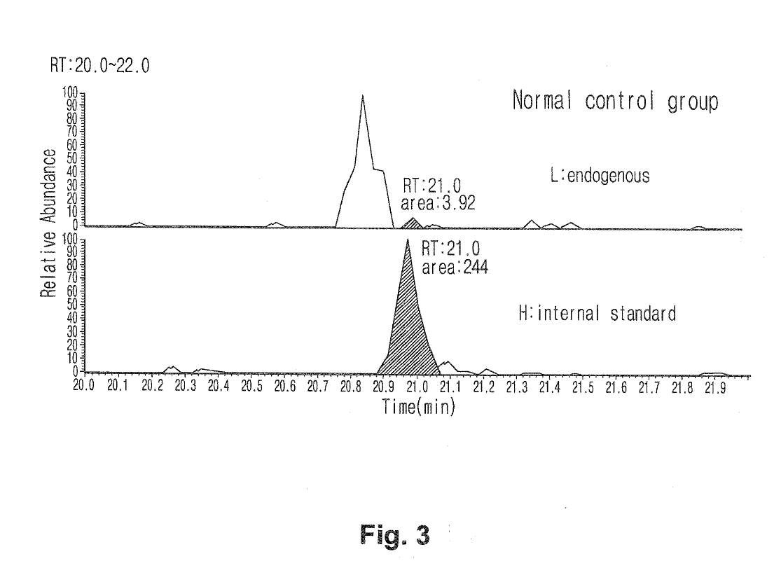

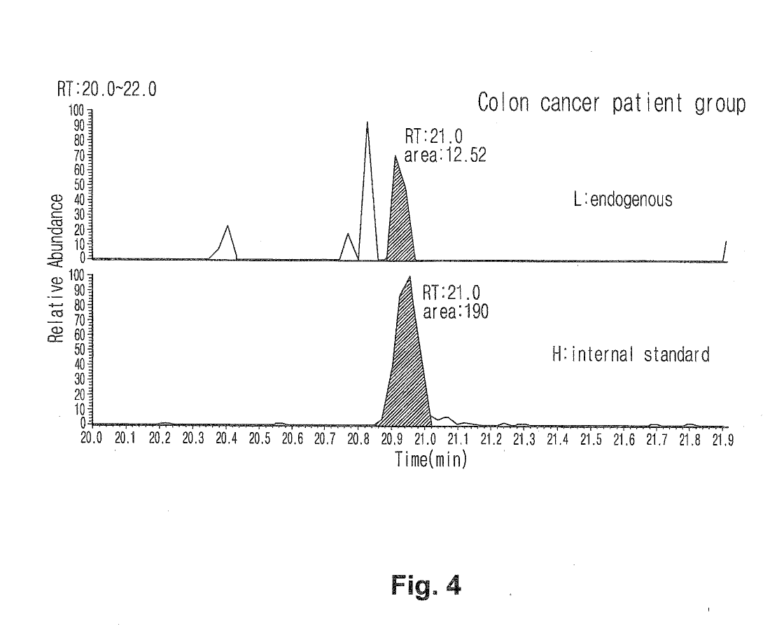

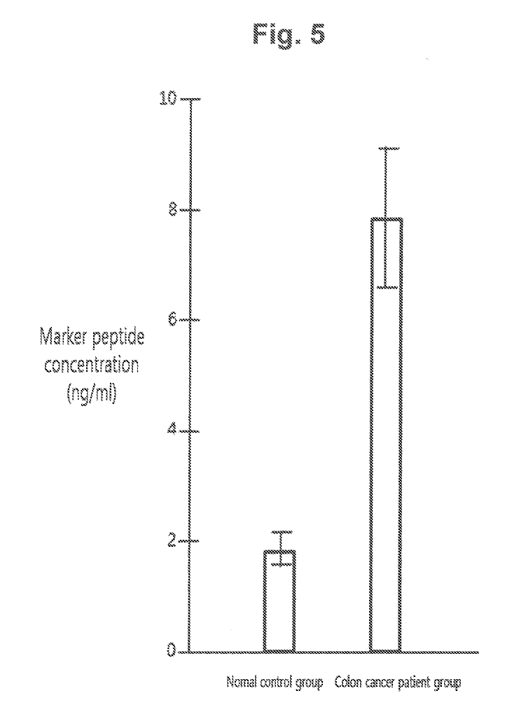

In a preferred embodiment of the present invention, the marker peptide having the peptide mass of 1232.6 was used to examine the blood sample to distinguish cancer patient group from normal group. Particularly, blood samples were obtained from patients diagnosed with colon cancer and from normal healthy people confirmed not to have any cancer related disease. Then, lectin selective protein samples were isolated from the blood samples. The isolated protein samples were hydrolyzed and as a result peptide samples were obtained from both cancer patient group and normal group. The target marker peptide was concentrated from the peptide samples, followed by LC/MRM with the marker peptide by using a peptide antibody. As a result, as shown in FIG. 3 and FIG. 4, the concentration of the marker peptide was significantly lowered in the normal group, compared with that in the colon cancer patient group (FIG. 3 and FIG. 4). And as shown in Table 3 and FIG. 5, the concentration of the marker peptide in the colon cancer patient group was approximately 3.4 times higher than that in the normal control group (FIG. 5).

Therefore, the marker peptide (peptide mass: 1232.6) screened and confirmed by using lectin in this invention showed high specificity to colon cancer and is confirmed to be very useful to distinguish colon cancer patients from normal people simply via blood analysis.

In conclusion, the marker peptides screened by the method of the present invention can be used as marker peptides for diagnosis, prognosis, or verification of cancer using human blood samples. In addition, quantitative analysis with a combination of at least one of those marker peptides can produce highly reliable results.

The present invention also provides a kit for cancer diagnosis comprising an antibody or a combination of antibodies specifically binding to one or more marker peptides selected from the group consisting of the marker peptides of the present invention.

The present invention also provides a use of the antibody or the combination of antibodies specifically binding to one or more marker peptides selected from the group consisting of the marker peptides of the present invention for the preparation of the kit for cancer diagnosis.

In this invention, the marker peptide is preferably the polypeptide having one of the amino acid sequences each represented by SEQ. ID. NO: 1-NO: 5, but not always limited thereto.

In this invention, the marker peptide is the one selected from the group consisting of the peptides each having the molecular weight of 2143.0, 1905.9, 1288.6, 1232.6, and 992.5, but not always limited thereto.

In this invention, the cancer is preferably the one selected from the group consisting of colon cancer, liver cancer, stomach cancer, lung cancer, uterine cancer, breast cancer, prostatic cancer, thyroid cancer, and pancreatic cancer, but not always limited thereto.

In this invention, the said kit facilitates monitoring, diagnosing, or screening of cancer by detecting quantitative changes in marker peptides generated by treating a sample obtained from a subject with a hydrolase.

In this invention, the said kit can additionally include the marker peptides or their isotope-labeled peptides as standard materials.

In this invention, the antibody usable in the kit includes polyclonal antibody, monoclonal antibody, and epitope linkable fragment, etc. The polyclonal antibody can be produced by the conventional method comprising the steps of injecting one of the peptide markers to an animal; drawing blood from an animal; and obtaining serum containing the antibody. Such polyclonal antibody can be produced using a host animal selected from the group consisting of goat, rabbit, sheep, monkey, horse, pig, cow, dog, etc, and purified by the conventional method well-known to those in the art. The monoclonal antibody can be prepared by any technique that can provide antibody molecules through continuous culture of cell line, which is exemplified by hybridoma technique, human B-cell hybridoma technique, and EBV-hybridoma technique (Kohler G et al., Nature 256:495-497, 1975; Kozbor D et al., J Immunol Methods 81:31-42, 1985; Cote R J et al., Proc Natl Acad Sci 80:2026-2030, 1983; and Cole S P et al., Mol Cell Biol 62:109-120, 1984), but not always limited thereto. Also, the antibody fragment containing the specific binding site to one of those peptide markers can be prepared (Huse W D et al., Science 254: 1275-1281, 1989). The method to prepare such peptide specific antibody having specific sequence is well known to those in the art.

In this invention, the antibody used in the kit of the present invention can be fixed on a solid substrate to make post-steps such as washing or isolation easy. The solid substrate herein is exemplified by synthetic resin, nitrocellulose, glass board, metal board, glass fiber, microsphere, and microbead, but not always limited thereto. The synthetic resin herein is exemplified by polyester, polyvinyl chloride, polystyrene, polypropylene, PVDF, and nylon, but not always limited thereto.

In this invention, the sample obtained from a subject is contacted with the antibody binding specifically to one of the peptide markers fixed on the solid substrate. At this time, the sample can be diluted properly before the contact.

In this invention, after contacting the sample obtained from a subject with the antibody binding specifically to one of the peptide markers fixed on the solid substrate, unbound proteins are removed by washing, followed by detection of the marker peptides.

In this invention, the kit of the present invention can additionally include an antibody for detection binding specifically to the said peptide marker. The antibody for detection herein can be a conjugate labeled with coloring enzyme, fluorescent material, radio-isotope, or colloid, etc, and preferably a secondary antibody binding specifically to the said marker, but not always thereto. The coloring enzyme herein can be peroxidase, alkaline phosphatase, or acid phosphatase (ex: horseradish peroxidase), but not always limited thereto. The fluorescent material herein can be fluorescein carboxylic acid (FCA), fluorescein isothiocyanate (FITC), fluorescein thiourea (FTH), 7-acetoxycourmarin-3-yl, fluorescein-5-yl, fluorescein-6-yl, 2′,7′-dichlorofluorescein-5-yl, 2′,7′-dichlorofluorescein-6-yl, dihydrotetramethylrhosamine-4-yl, tetramethylrhodamine-5-yl, tetramethylrhodamine-6-yl, 4,4-difluoro-5,7-dimethyl-4-bora-3 a,4a-deaza-s-indacene-3-ethyl or 4,4-difluoro-5,7-diphenyl-4-bora-3a,4a-deaza-s-indacene-3-ethyl, but not always limited thereto.

In this invention, the said kit can additionally include a substrate to be reacted with coloring enzyme, and washing fluid or eluent to eliminate unbound proteins and to keep conjugated peptide markers only.

The present invention also provides a biochip for cancer diagnosis comprising a biomolecule specifically binding to one or more marker peptides selected from the group consisting of the marker peptides of the present invention is integrated on the solid substrate.

The present invention also provides a use of the biomolecule specifically binding to one or more marker peptides selected from the group consisting of the marker peptides of the present invention for the preparation of the biochip for cancer diagnosis.

In this invention, the marker peptide is preferably the polypeptide having one of the amino acid sequences each represented by SEQ. ID. NO: 1-NO: 5, but not always limited thereto.

In this invention, the marker peptide is preferably the one selected from the group consisting of the peptides each having the molecular weight of 2143.0, 1905.9, 1288.6, 1232.6, and 992.5, but not always limited thereto.

In this invention, the cancer is preferably the one selected from the group consisting of colon cancer, liver cancer, stomach cancer, lung cancer, uterine cancer, breast cancer, prostatic cancer, thyroid cancer, and pancreatic cancer, but not always limited thereto.

In this invention, the said biochip facilitates monitoring, diagnosing, or screening of cancer by detecting quantitative changes in marker peptides generated by treating a sample obtained from a subject with a hydrolase.

In this invention, the biomolecule is preferably an antibody or an aptamer, but not always limited thereto. The said biomolecule indicates not only a small molecule such as primary metabolite, secondary metabolite and natural substance but also an organic molecule produced by living organism such as protein, polysaccharide and nucleic acid. The aptamer herein indicates oligonucleotide or peptide binding to the specific target molecule.

In this invention, the solid substrate is preferably selected from the group consisting of plastic, glass, metal, and silicon, but not always limited thereto.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Sample Preparation

To overexpress N-acetylglucosaminyltransferase (GnT-V), the glycosyltransferase overexpressed in cancer cells, human colon cancer cells were transfected with MGAT5, resulting in the preparation of stable transfectant cells. Then, the GTN-V overexpressing cells (GnT-V-treated cells) and the control cells were cultured. Equal amounts of culture fluids obtained from both group were concentrated until the volume reached 2 ml respectively. The concentrated samples were reduced using 14 mM β-mercaptoethanol, followed by desalting. One mg of the desalted protein was loaded to L-PHA-avidin-agarose beads, to which phosphate-buffered saline (PBS) was added. The mixture stood at 4° C. for 12 hours. The lectin conjugated protein was washed with PBS three times, and then the protein was separated from lectin by using 6 M urea. The obtained protein was 10-fold diluted with 50 mM ammonium bicarbonate, followed by hydrolysis using 10 ug of trypsin for overnight at 37° C. with 1 mM CaCl₂. The hydrolyzed peptide was dried under reduced pressure to give 100 ul liquid. For the sample that had not been through lectin separation, 100 ug of each protein obtained from reduction and desaltation of the concentrated culture fluid was hydrolyzed. The hydrolyzed peptide was dried under reduced pressure to give 100 ul liquid.

### Example 2

### Selection of Marker Candidates Via Peptide Analysis

HPLC (high-performance liquid chromatography) was performed using trap column (C18, 5 um, 300×5 mm) and analytical column (C18, 5 um, 75 um×10 cm) to analyze the samples prepared in Example 1, followed by LC/ESI-MS/MS using LTQ-FT mass spectrometer (Thermo Finnigan), the electrospray ionization (ESI) mass spectrometer. Each sample protein was hydrolyzed by using trypsin to obtain peptides. Some of the prepared peptide samples were 10-fold diluted, which proceeded to liquid chromatography connected to the mass spectrometer by 10 μl.

Based on the results of the mass spectrometry, various proteins were qualified from the samples obtained from the GnT-V treated cell line and the normal control cell line by using search engines such as MASCOT and SEQUEST. The relative amount of each protein existed in each sample was calculated based on the frequency of qualitative analysis of each protein. Significant proteins were screened from the peptides obtained by LC/ESI-MS/MS and their analysis frequencies. Glycosylation of the screened protein and relevancy of the protein with cancer were investigated by using protein database including NCBInr DB, etc, related papers and descriptions, etc. As a result, candidate glycoproteins implying high chance of relevancy with cancer were selected. It was investigated whether or not the selected glycoprotein candidates could be used as cancer markers by quantitative analysis using multiple reaction monitoring mass spectrometry (MRM MS) with the peptides obtained by hydrolyzing the protein.

Among the marker peptides (Table 1) generated by hydrolysis of TIMP metallopeptidase inhibitor 1 (TIMP1, MW: 20.7 k, Theoretical pl: 8.47) believed as a promising cancer marker, the present inventors have confirmed the possibility of using the peptide having the peptide mass of 1232.6 as a marker by MRM MS.

TABLE 1

| Peptide | | |
|---|---|---|
| Number | Peptide | |
| (No.) | Mass | Peptide Sequence (SEQ. ID. NO) |
| 1 | 2143.0 | LQSGTHCLWTDQLLQGSEK |
| | | (SEQ. ID. NO: 1) |
| 2 | 1905.9 | FVYTPAMESVCGYFHR (SEQ. ID. NO: 2) |
| 3 | 1288.6 | EPGLCTWQSLR (SEQ. ID. NO: 3) |
| 4 | 1232.6 | GFQALGDAADIR (SEQ. ID. NO: 4) |
| 5 | 992.5 | SEEFLIAGK (SEQ. ID. NO: 5) |

The marker peptides shown in Table 1 were all generated from one glycoprotein by hydrolysis, so that all the peptides of Table 1 can be used as a representative of the glycoprotein in mass spectrometry.

### Example 3

### Confirmation of Marker Glycoprotein by Mass Spectrometry

Peptide samples obtained from the protein of each subject by hydrolysis using trypsin were added at the volume of 50 fmol to make the total volume 50 μl. 4 samples were prepared; 2 of them were through lectin separation and the other 2 were not finished with lectin separation. The standard material labeled with target peptide isotope was the synthetic standard material that had the identical amino acid sequence with the target peptide (marker peptide) but was different in peptide mass. This material was used as an internal standard material in MRM MS. In quantitative mass spectrometry, calibration curve was made according to the changes of concentrations of the target peptide and the synthetic standard material under the same analysis conditions.

A part (10 μl) of each prepared sample (50 μl) was used for LC/MRM MS. Quantitative analysis was performed in triplicate with the marker peptide (peptide mass, 1232.6) by MRM MS and the results are shown in Table 2.

TABLE 2

| | Sample | av. (fmol) | RSD (%) |
|---|---|---|---|
| With lectin | Control | 0.31 | 12.9 |
| separation | GnT-V treated | 3.90 | 8.4 |
| Without | Control | 28.75 | 9.3 |
| lectin | GnT-V treated | 47.46 | 2.0 |
| separation | | | |

As shown in Table 2, the results obtained from the triplicated quantitative analysis with each sample demonstrated very satisfactory relative standard deviations. These results indicate that the quantitative analysis for the marker peptide of the present invention is very stable and thus the marker peptide is highly reliable. Even at the hundreds of attomol level, the marker peptide can be quantified with low standard deviation but with high sensitivity. In particular, when comparing two samples obtained by performing lectin separation (GnT-V treated group and control group), detected amount of the marker peptide in the GnT-V treated group was 12.5 fold higher (FIG. 1) than that of the control group. However, difference of the marker peptide amount in the two samples obtained by not-performing lectin separation was not that much (FIG. 2). The above results indicate that the marker peptide screened and confirmed by using lectin (peptide mass, 1232.6) has high specificity against cancer cells overexpressing GnT-V. In addition, liability to the marker glycoprotein of the present invention can be increased by using a combination of one or more marker peptides listed in Table 1 for quantitative mass spectrometry.

### Example 4

### Confirmation of Marker Glycoprotein by Using Clinical Blood Sample

Pooled cancer plasma was prepared by mixing blood samples obtained from 10 patients clinically diagnosed as colon cancer. Pooled control plasma was also prepared by mixing blood samples obtained from 10 healthy people who were clinically confirmed not to have any kind of cancer. Lectin selective protein samples were separated from both blood samples of the cancer patient group and the control group by the same manner as described in Example 1, followed by hydrolysis to prepare peptide samples of the cancer patient group and the control group. The standard material labeled with isotope of the marker peptide (peptide mass, 1232.6) represented by SEQ. ID. NO: 4 (Table 1) was added to each sample equally, and used as an internal control material for quantitative analysis.

The target protein exists in blood at a very low concentration, so that the target marker peptide cannot be detected from the prepared peptide sample by MRM right away. Therefore, the target marker peptide was concentrated from each of the prepared peptide sample by using the target marker peptide (SEQ. ID. NO: 4) selective peptide monoclonal antibody (anti-peptide antibody). At this time, the monoclonal anti-peptide antibody can be fixed directly on polymer solid or magnetic solid for the convenience of experiment, or fixed indirectly by using avidine-biotin linker, etc. In this invention, the monoclonal anti-peptide antibody was directly fixed on magnetic beads.

The marker peptide was concentrated from the hydrolyzed peptide sample by using the monoclonal anti-peptide antibody, followed by LC/MRM. FIG. 3 and FIG. 4 are examples of chromatogram illustrating the transition of the marker peptide (SEQ. ID. NO: 4) obtained by LC/MRM. The endogenous marker peptide and the internal standard were simultaneously eluted on chromatography in both groups of colon cancer patient and normal control. In the normal control group, concentration of the endogenous marker peptide was significantly lower than that in the colon cancer patient group. The quantitative analysis was repeated. As a result, as shown in Table 3 and FIG. 5, concentration of the marker peptide in the colon cancer patient group was approximately 4.3 fold higher than that in the control group (Table 3 and FIG. 5).

TABLE 3

| | | | MRM | | |
|---|---|---|---|---|---|
| | | | Quantitative | Marker | |
| | Blood | Internal | Value | Peptide | |
| | Sample | Control | (av. | Conc. | RSD |
| Sample | (ul) | (fmol) | fmol/ul) | (av. ng/ml) | (%) |
| Normal | 30 | 100 | 0.062 | 1.8 | 18.3 |
| Control | | | | | |
| Group | | | | | |
| Colon | 30 | 100 | 0.261 | 7.8 | 22.4 |
| Cancer | | | | | |
| Group | | | | | |

### Example 5

### Confirmation of TIMP1 Derived Marker Peptide as Lung Cancer Marker

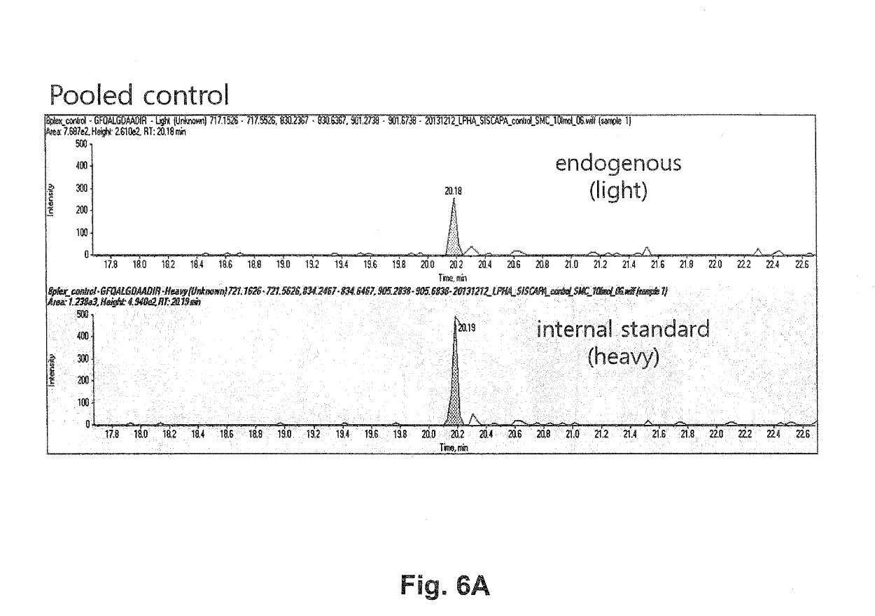

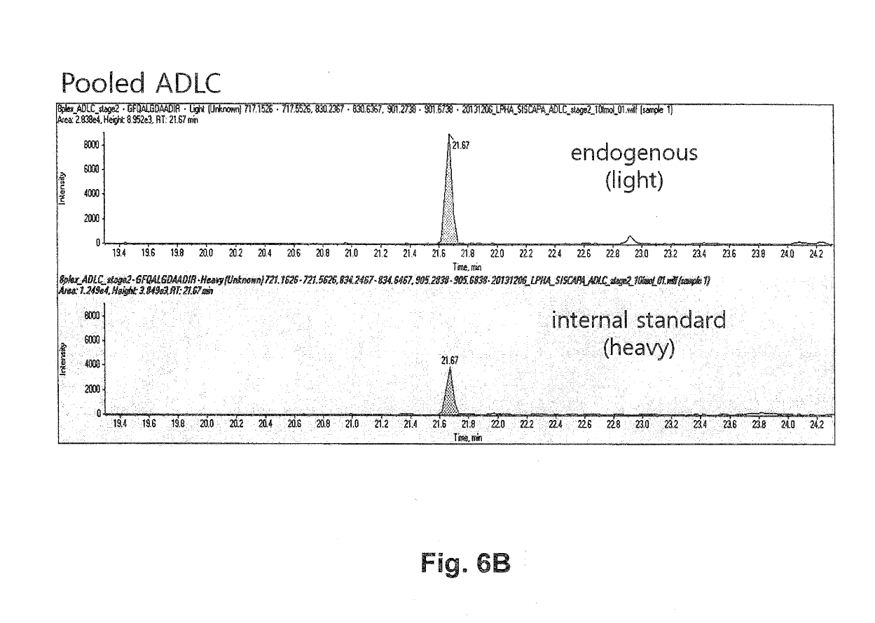

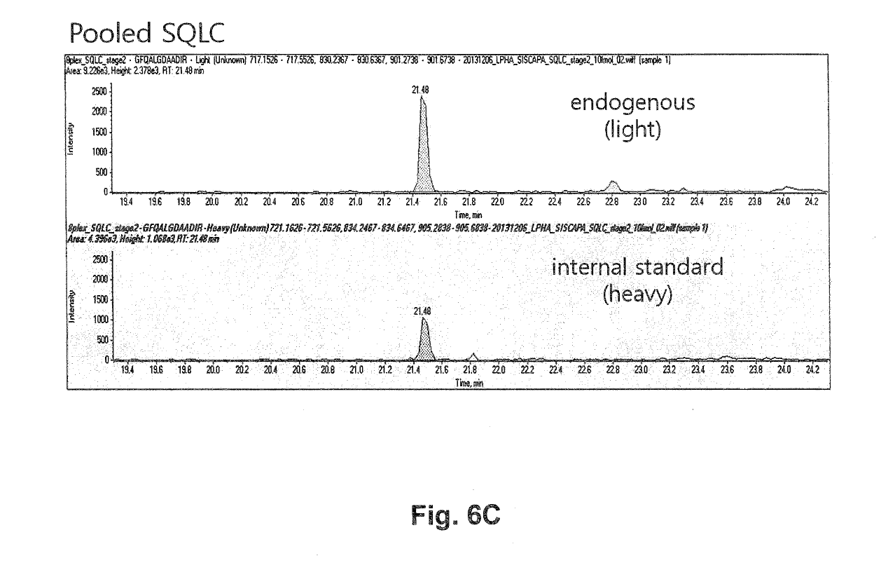

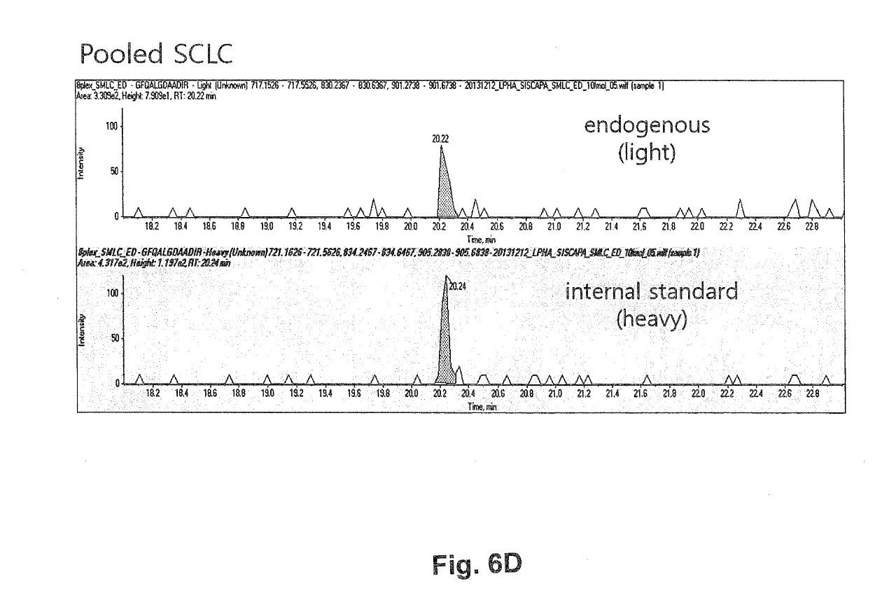

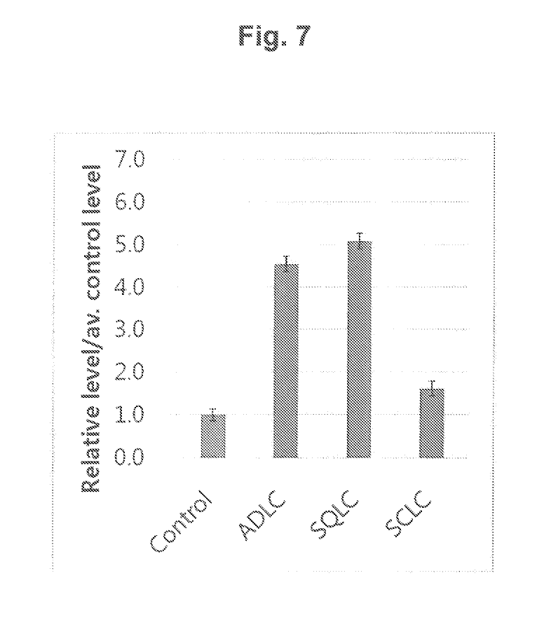

Blood samples collected from clinically diagnosed lung cancer patients were used to analyze with the method of Example 4, to develop lung cancer marker glyprotein TIMP metallopeptidase inhibitor 1 (TIMP1, MW: 20.7 k, Theoretical pl: 8.47) for the diagnosis of lung cancer. By pooling 10 cases of blood samples which were clinically diagnosed adenocarcinoma lung cancer (ADLC), squamous-cell carcinoma lung cancer (SQLC), and small-cell lung cancer (SCLC), respectively, the pooled ADLC serum sample, pooled SQLC serum sample and pooled SCLC serum sample were prepared. Also by pooling blood samples from 10 subjects with no cancer diagnosis opinion, pooled control serum sample was prepared. Quantitative analysis was performed three times on the marker peptide of SEQ ID NO: 4 (peptide mass, 1232.6, Table 4) according to Example 4 (FIGS. 6A-D). The result of the quantitative analysis was summarized into Table 4. The analysis result of Table 4 indicates that it is possible to quantitatively identify lung cancer group (ADLC and SQLC groups) from the normal control using the developed marker (FIG. 7). It is particularly confirmed that by using the marker peptide according to the present invention, it is possible to identify adenocarcinoma and squamous-cell carcinoma subtype from the small-cell lung cancer subtype.

TABLE 4
Analysis on lung cancer blood samples using TIMP1-
derived marker peptide SEQ ID NO: 4

| | | | | Av. | | |
|---|---|---|---|---|---|---|
| | | Internal | | relative | | |
| | Blood | Standard | Av. area | level | | |
| Serum | Sample | (H) | ratio | to | | RSD |
| class | (ul) | (fmol) | (L/H) | av. control | SD | (%) |
| Control | 48 | 10 fmol | 0.494 | 1.00 | 0.14 | 13.7 |
| ADLC | 48 | 10 fmol | 2.243 | 4.54 | 0.18 | 3.9 |
| SQLC | 48 | 10 fmol | 2.507 | 5.08 | 0.19 | 3.7 |
| SCLC | 48 | 10 fmol | 0.798 | 1.60 | 0.17 | 10.5 |

The above results indicate that the marker peptide represented by SEQ. ID. NO: 4 (peptide mass, 1232.6) which has been screened and confirmed by using lectin has high specificity to colon cancer and lung cancer.

Therefore, it was confirmed that the marker peptide of the present invention can be effectively used for the distinguishment of the colon cancer and lung cancer patient group from the normal healthy group by analyzing blood samples. In addition, the reliability of the marker peptide of the present invention can be much increased by using a combination of one or more marker peptides listed in Table 1 which are simultaneously generated by hydrolysis of a single marker glycoprotein.

### INDUSTRIAL APPLICABILITY

As explained hereinbefore, the present invention provides a method for screening a cancer marker, a method for confirming the same, and a method for cancer diagnosis using the said marker peptide.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for identifying a subject having lung cancer or having a high risk for developing lung cancer, comprising:
a) contacting blood from a test subject with one or more lectins;
b) hydrolyzing glycoproteins that bind one or more lectins with one or more enzymes to produce peptides derived from a glycoprotein, wherein the hydrolysis is conducted in solution;
c) concentrating the glycoprotein-derived hydrolysate of step b) by contacting with a peptide-selective monoclonal antibody or a combination of peptide-selective antibodies that specifically binds a peptide derived from the glycoprotein of step b);
d) analyzing the antibody-bound peptides from step c) to determine the level of target peptides having the amino acid sequences represented by SEQ ID NO:1, SEQ ID NO:2, SEQ ID NO:3, SEQ ID NO:4, and SEQ ID NO:5 wherein the target peptides were derived from a glycoprotein TIMP1 having a cancer cell-specific glycan chain; and
e) identifying the test subject as having lung cancer or having a high risk for developing lung cancer when the level of the target peptides from step d) is greater than the level of the corresponding target peptides in a normal subject.

2. The method of claim 1, wherein the lectin is selected from the group consisting of ConA, WGA, Jacalin, SNA, AAL, L-PHA, PNA, LCA, ABA, DBA, DSA, ECA, SBA, SSA, UEA, VVL, and BPL.

3. The method of claim 1, wherein the one or enzyme is selected from the group consisting of Arg-C, Asp-N, Glu-C, Lys-C, chymotrypsin, and trypsin.

4. The method of claim 1, wherein the step of analyzing comprises performing LC/mass spectrometry.

5. The method of claim 1, wherein lung cancer is adenocarcinoma lung cancer (ADLC) or squamous-cell carcinoma lung cancer (SQLC)

6. A method for identifying a subject having lung cancer or having a high risk for developing lung cancer, comprising:
a) contacting blood from a test subject with one or more lectins;
b) hydrolyzing glycoproteins that bind one or more lectins with one or more enzymes to produce peptides derived from a glycoprotein, wherein the hydrolysis is conducted in solution;
c) concentrating the glycoprotein-derived hydrolysate of step b) by contacting with a peptide-selective monoclonal antibody or a combination of peptide-selective antibodies that specifically binds a peptide derived from the glycoprotein of step b);
d) analyzing the antibody-bound peptides from step c) to determine the level of target peptides having a molecular weight of 2143.0, 1905.9, 1288.6, 1232.6 and 992.5 Da, wherein the target peptides were derived from a glycoprotein TIMP1 comprising a cancer cell-specific glycan chain; and,
e) identifying the test subject as having lung cancer or having a high risk for developing lung cancer when the level of the target peptides from step d) is greater than the level of the corresponding target peptides in a normal subject.

7. The method of claim 6, wherein the lectin is selected from the group consisting of ConA, WGA, Jacalin, SNA, AAL, L-PHA, PNA, LCA, ABA, DBA, DSA, ECA, SBA, SSA, UEA, VVL, and BPL.

8. The method of claim 6, wherein the one or enzyme is selected from the group consisting of Arg-C, Asp-N, Glu-C, Lys-C, chymotrypsin, and trypsin.

9. The method of claim 6, wherein the step of analyzing comprises performing LC/mass spectrometry.

10. The method of claim 6, wherein lung cancer is adenocarcinoma lung cancer (ADLC) or squamous-cell carcinoma lung cancer (SQLC).
